(19) United States
(12) Patent Application Publication
(10) Pub. No.: US 2008/0319006 A1
(43) Pub. Date: Dec. 25, 2008

(54) Nebulizer Formulation

(75) Inventors: Ian Gardner Cameron McAffer, Kent (GB); Peter Ernest Tasko, Kent (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: Breath Limited , a corporation

(21) Appl. No.: 11/815,085

(22) PCT Filed: Jan. 30, 2006
(86) PCT No.: PCT/GB2006/000309

§ 371 Date: Apr. 1, 2008

(30) Foreign Application Priority Data
Jan. 31, 2005 (GB) 0501956.7

(51) Int. Cl. A61K 31/439; A61P 11/00

(52) U.S. Cl. 514/304

(57) ABSTRACT

A nebulizer formulation contains levalbuterol and ipratropium in about 2 ml or less of saline and is used for treatment of COPD and asthma and other airways diseases and disorders with increased patient compliance.

### BACKGROUND OF THE INVENTION

The present invention relates to a nebulizer formulation, in particular a nebulizer formulation and a method of treatment for diseases such as Chronic Obstructive Pulmonary Disease (COPD) and asthma using the formulation.

Nebulizers provide a means of administering drugs to the airways of a patient whilst the patient breathes at an approximately normal rate. They are particularly suitable for patients who are unable, whether due to age or injury or otherwise, to inhale at the much higher rates required for administration of drugs via metered dose inhalers or dry powder inhalers and for patients who cannot for whatever reason coordinate the activation of the metered dose inhaler with their inhalation of breath. The nebulizer apparatus creates a vapour containing drug particles and the patient breathes the vapour via a mouthpiece or mask attached to the nebulizer. Typically, nebulizers are used to deliver drugs for the treatment of airways disorders such as asthma and COPD.

According to the U.S. Centers for Disease Control and Prevention, COPD is currently the fourth leading cause of death in the U.S. (behind heart disease, cancer and stroke), claiming the lives of in excess of 100,000 Americans annually. An estimated 16 million Americans have been diagnosed with some form of COPD, and as many as 16 million others have the condition but have not yet been diagnosed. COPD is hence regarded as a major and growing health care threat in the U.S. and throughout the rest of the world.

A known formulation for treatment of COPD comprises albuterol (also known as salbutamol) and ipratropium in an ampoule containing 3.0 ml of solution, and is described in WO 03/037159 and the equivalent U.S. Pat. No. 6,632,842. In use, the contents of the ampoule are poured into the chamber of the nebulizer and the patient then breathes the vapour generated until the ampoule contents are used. Treatment is typically required up to 4 times per day, at regular intervals.

Low patient compliance is a known problem with nebulized drugs generally, as the period of nebulizing required to administer a dose is long, typically tens of minutes, perhaps half-an-hour for a typical dose. Children and adults can become bored during this period. Patients who stop nebulizing prematurely do not receive a full dose. This can in turn lead to further reduced patient compliance as the inadequate dose fails to provide adequate therapy, discouraging further use.

It is an object of the present invention to provide formulations and their uses which overcome or at least ameliorate one or more of the above-identified problems.

### SUMMARY OF THE INVENTION

The present invention provides novel nebulizer formulations, suitable for treatment of COPD, asthma and other conditions associated with reversible obstruction of the airways. The formulations can be utilized in a variety of known nebulizer apparatus with reduced wastage of ingredients and/or increased patient compliance.

The invention provides a method of treatment of COPD, asthma and other conditions associated with reversible obstruction of the airways comprising administering, via a nebulizer, a formulation containing both levalbuterol and an anticholinergic agent in a pharmaceutically acceptable carrier.

More specifically, the present invention provides a method of treatment of COPD, asthma and other conditions associated with reversible obstruction of the airways, comprising providing a nebulizer and an ampoule containing not more than 2.2 ml of a formulation comprising levalbuterol and an anticholinergic agent, such as ipratropium, in a pharmaceutically acceptable carrier, and administering the formulation using the nebulizer.

A filled ampoule of the invention contains a formulation of levalbuterol and an anticholinergic agent, e.g. ipratropium, and a more specific ampoule contains a nebulizer formulation, wherein the formulation contains from 1.0 to 1.5 mg of levalbuterol, and from 0.40 to 0.70 mg of ipratropium, in a total volume of from 0.3 to 5 ml.

Also provided by the invention is a method of increasing patient compliance in use of a nebulizer formulation, comprising providing a nebulizer, and an ampoule containing said formulation, wherein the formulation comprises levalbuterol and an anticholinergic agent in a pharmaceutically acceptable carrier and wherein the ampoule contains not more than 2.2 ml and not less than 0.3 ml of said formulation, and administering the formulation using the nebulizer.

A kit of the invention comprises a formulation of the invention with instructions on how to use it.

### DETAILED DESCRIPTION OF THE PREFERRED EMBODIMENTS

The invention enables use of nebulizer formulations without the lower limit on volume typically associated with the art. The beta agonist is formulated with and administered with an anticholinergic agent, suitably selected from ipratropium, tiaproprium, cromolyn sodium and other anticholinergic agents.

Reference to these active agents herein is intended to refer also to pharmaceutically acceptable derivatives thereof, such as but not limited to salts, esters, enol ethers, enol esters, acids, bases, solvates, hydrates or prodrugs thereof. Ipratropium is an anticholinergic agent and has a bronchodilator effect, and can thus aid delivery of, and act in synergy with the bronchodilator effect of, levalbuterol. Reference to ipratropium thus includes, but is not limited to, any form of ipratropium which has an anticholinergic effect in patients suffering from COPD, including, but not limited to, all automatic forms, enantiomer forms, stereoisomer, anhydrides, acid addition salts, base salts, solvates, analogues and derivatives of ipratropium.

Several acceptable salts of ipratropium exist and for the invention these may include, but are not limited to, halide salts such as bromide, chloride and iodide, described for example in U.S. Pat. No. 3,505,337, which is incorporated herein by reference.

A method of treatment of COPD or asthma using the teachings of the invention comprises administering to a human patient, via a nebulizer, a formulation containing effective amounts of both levalbuterol and ipratropium in a pharmaceutically acceptable carrier, and it is expected, using this ampoule formulation, to achieve improved acceptance of the medicine and better patient compliance.

A further anticipated advantage is that the concentration of the active ingredients can be increased, with the result that ampoules of the invention can contain reduced volume whilst retaining substantially the same unit dosage of drug to be administered. In one embodiment of the invention overall ampoule concentration (ie. including active and inactive ingredients) is substantially the same as in previous formulations but ampoule volume is approximately half. With reduced volume, there is reduced nebulizing time. This will further improve patient compliance as there is less time e.g. for patients to become bored or distracted whilst using the nebulizer.

In preferred embodiments of the invention, both ampoule volume is reduced, enabling reduced nebulising time, and ampoule concentration is reduced.

The compositions provided herein are used for treating, preventing, or ameliorating one or more symptoms of a bronchoconstrictive disorder or disease in a human subject. In one embodiment, the method includes nebulizer administration to a subject of an effective amount of a composition containing levalbuterol and an anticholinergic, whereby the disease or disorder is treated or prevented.

The invention relates in particular to formulations for treatment of COPD and asthma, including, but not limited to, chronic bronchitis, emphysema, and associated cor pulmonale (heart disease secondary to disease of the lungs and respiratory system) with pulmonary hypertension, right ventricular hypertrophy and right heart failure, and also bronchial asthma, allergic asthma and intrinsic asthma, e.g., late asthma and airway hyper-responsiveness.

The formulations of the present invention are designed for administration by nebulizer. A nebulized solution is one dispersed in air to form an aerosol, and a nebulizer generates very fine liquid droplets suitable for inhalation into the lung. Nebulizers typically use compressed air, ultrasonic waves, or a vibrating mesh to create a mist of the droplets and may also have a baffle to remove larger droplets from the mist by impaction. A variety of nebulizers are available for this purpose, such as ultrasonic nebulizers, jet nebulizers and breath-actuated nebulizers. In use, mouthpieces or masks are typically attached to a patient to aid delivery of the nebulized solution.

In preferred embodiments of the invention, formulations are for delivery with and patients are treated using a high efficiency nebulizer, in particular one that can deliver at least 15%, preferably at least 25%, more preferably at least 35% of the drug substance to the patient's lungs.

In specific embodiments of the invention, formulations are delivered using a high efficiency jet nebulizer, a high efficiency ultrasonic nebulizer or a high efficiency vibrating mesh nebulizer, use of these devices enabling and/or enhancing the use of the reduced volume formulations of the invention. Jet nebulizers are particularly preferred, and one example is the PARI LC Plus (registered trade mark, Pari GmbH, Germany) nebulizer.

The invention is of particular application to COPD. Specifically, a method of treatment of COPD according to the present invention hence comprises:

(1) providing:
a) a nebulizer, and
b) an ampoule containing not more than 2.2 ml of a formulation comprising levalbuterol and ipratropium in a pharmaceutically acceptable carrier, and

(2) administering the formulation using the nebulizer.

Preferably, the invention provides and uses ampoules which contain not less than 0.5 ml of formulation, more preferably about 1.0 to 2 ml of said formulation, and very preferably about 1.5 ml to 2 ml of said formulation. These reduced volumes can lead to significant reductions in treatment times, with the expected advantages explained.

Formulations and compositions of the invention generally comprise a pharmaceutically acceptable carrier. The carrier is preferably a liquid carrier. Further, the carrier preferably comprises water and may comprise other components.

A filled ampoule of the invention contains a formulation of levalbuterol and ipratropium. This is generally in a pharmaceutically acceptable carrier and buffered for human use to a pH of about 3.5-5.5. The formulations of the examples are buffered to about pH 4. The levalbuterol is used in the examples as the HCl salt, though other salts are suitable, and the ipratropium is generally used as its HBr salt, though again other salts are suitable. HCl is conveniently used for pH adjustment. The formulations are free of preservative, which is an advantage as some preservatives can be associated with bronchoconstrictor effects—the opposite effect to that required by the formulation. Water is used to provide the carrier, and water for injection is preferred due to its purity.

One or more tonicity adjusting agents may be added to provide the desired ionic strength. Tonicity adjusting agents for use herein include those which display no or only negligible pharmacological activity after administration. Both inorganic and organic tonicity adjusting agents may be used. Compositions of the invention can also include excipients and/or additives. Examples of these are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In one embodiment, the complexing agent is EDTA. Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof.

Formulations as described in this invention can be readily prepared by a person of skill in the art. In one method, a solution of NaCl is prepared with concentration approximately 9 g/l. To this are added ipratropium bromide to a concentration as desired, but typically about 0.25 mg/ml and levalbuterol, again to the concentration desired but typically about 0.625 mg/ml. HCl is then added to give a final pH of about 4.0. This formulation can be filled into ampoules using blow fill seal technology (described in more detail below) to yield ampoules with the required extractable volume of formulation.

Reference to an ampoule with a specified volume and to the extractable volume of an ampoule refer to the volume of solution that can be extracted from the ampoule in normal use, e.g. by breaking it open and pouring out the contents without actively flushing the ampoule or carrying out scientific extraction methods. There is in addition some tolerance in the volumes recited, as filling machines vary in their accuracy. By way of illustration, “an ampoule containing 3 ml of solution” and a “3 ml ampoule”, say, both refer to an ampoule which contains about 3.1 to about 3.2, generally about 3.15, ml of solution and which when opened and poured into the nebulizer results in approximately 3 ml of solution being transferred into the nebulizer. Hence, the volumes recited refer to the amount of solution that can be readily extracted from the ampoule rather than the amount the ampoule is filled with.

Preferred embodiments of the invention provide methods and ampoules for adults, for whom a typical dose of levalbuterol is about 1.25 milligrams. A typical dose of ipratropium is about 0.5 milligrams. Ampoules thus suitably contain such amounts of the active ingredients in their extractable volume.

Preferably, ampoules of the invention have reduced volume, containing 2.2 ml or less of said formulation, preferably 2.0 ml or less of said formulation or about 1.0 to 2 ml of said formulation. Specific embodiments of the invention, set out in detail below, provide ampoules of about 2 ml. Other suitable ampoule volumes are about 1.5 ml, about 1.0 ml and about 0.5 ml.

In use of the formulations of the invention, it is possible to deliver a sufficient dose of ipratropium and levalbuterol in a shorter period of time than is necessary for known combination formulations. Hence, the invention provides in another aspect a method of increasing patient compliance in use of a nebulizer formulation, comprising

(1) providing:
a) a nebulizer, and
b) an ampoule containing said formulation, wherein the formulation comprises levalbuterol and ipratropium in a pharmaceutically acceptable carrier and wherein the ampoule contains not more than 2.2 ml and not less than 0.3 ml of said formulation, and

(2) administering the formulation using the nebulizer.

The volume of the ampoule can be reduced following the teachings of the invention. However, there is a practical limit to the concentration of the contents of ampoules of the invention in as much as very small amounts of highly concentrated liquids are easily spilled and are not so easy to dispense accurately. In preferred methods and formulations, the ampoules contain not more than 2 ml and not less than 0.5 ml of said formulation.

The formulation used in the method typically contains from 0.75 to 2.0 mg of levalbuterol, preferably from 1.0 to 1.5 mg of levalbuterol. The formulation also typically contains from 0.25 to 1.0 mg of ipratropium, preferably from 0.40 to 0.70 mg of ipratropium. These formulations preferably have volumes of about 2.0 ml, about 1.5 ml, about 1.0 ml or about 0.5 ml.

A specific ampoule contains a nebulizer formulation of:
a) from 1.0 to 1.5 mg of levalbuterol, and
b) from 0.40 to 0.70 mg of ipratropium,
   in a volume of from 0.3 to 2.2 ml.

The total volume is preferably from 0.5 ml to 2 ml, and more preferably has a total volume of about 2 ml or about 1.5 ml.

In use of the invention, increased concentration of the active ingredients, ipratropium and levalbuterol, enables smaller volumes of the formulation to be used. Generally, the levalbuterol is present at about 0.57 mg/ml or higher, about 0.63 mg/ml or higher or about 0.83 mg/ml or higher. An ampoule with an extractable volume of 0.5 ml can contain levalbuterol at about 2.5 mg/ml. The ipratropium is generally present at a concentration of about 0.23 mg/ml or higher, about 0.25 mg/ml or higher or about 0.33 mg/ml or higher. An ampoule with an extractable volume of 0.5 ml can contain ipratropium at about 1 mg/ml.

Pharmaceutical compositions containing levalbuterol and an anticholinergic agent for administration via nebulization are hence provided. The compositions are preferably sterile filtered and filled in ampoules or vials, including unit dose vials, providing sterile unit dose formulations for use in a nebulizer.

In a further embodiment, the present invention provides a container containing a vial, comprising a single unit dose of a therapeutically effective amount of levalbuterol and ipratropium in a sterile solution, or a plurality of such vials. The extractable volume of each unit dose of a specific embodiment of the invention comprises about 1.25 mg of levalbuterol (or equivalent amount of a derivative thereof) and about 0.5 mg ipratropium bromide (or equivalent amount of a derivative thereof) in a sterile, aqueous solution. The solution contains sodium chloride at about 9 mg/ml to make the solution isotonic and hydrochloric acid to adjust pH of the solution to about 4.0. It is optional to include a chelating agent, such as EDTA. The volume is preferably about 2.0 ml or about 1.5 ml.

The invention additionally provides kits for use in treatment of the diseases described herein. The kits comprise:
(1) a container, containing a single unit dose of a therapeutically effective amount of levalbuterol plus an anticholinergic agent; and
(2) instructions on how the dose is to be used.

The single unit dose is suitably as described elsewhere herein in relation to formulations of the invention. The instructions instruct the patient as to how the dose should be used in conjunction with a nebulizer, such as how to open it and transfer its contents into the nebulizer, how to operate the nebulizer and for how long nebulizing should be continued to complete administration of the dose.

Kits of the invention can contain a plurality of single unit doses. One kit comprises at least 120 or at least 125 single unit doses, being designed to provide one month's worth of doses to be taken 4 times per day. Another kit comprises at least 25 or at least 28 single unit doses, designed for a week's supply at 4 per day. Other kits may usefully contain 30 or 60 single unit doses.

For embodiments of the invention in which the extractable volume is 2.2 ml or less, especially where the volume is 2.0 ml or 1.5 ml or lower, the instructions may explain that the present formulation can be administered in less time than a previously known formulation, such as a known 3 ml formulation, hence reinforcing this advantage of the invention and improving the prospects for increased patient compliance. Preferably, the instructions explain that the patient should continue administering the dose until the complete dose has been administered.

Formulations of the invention are suitable for filing into ampoules using “blow fill seal” (BFS) methods. The principle is that a plastic parison is extruded from polymer, formed into a container, filled and sealed in a single aseptic operation. BFS is now the preferred method for aseptic manufacture of ampoules due to the flexibility in container design, overall product quality, product output and low operational costs. Fill accuracies of better than ±5% have been demonstrated for container volumes as small as 0.5 ml and hence BFS is suitable for manufacture of ampoules according to the invention.

One BFS operation includes the multi-step process of blow moulding, aseptic filling and hermetic sealing of liquid products with fill volumes ranging from 0.1 ml to 1,000 ml, though for ampoules volumes in the range 0.5 ml to 5 ml are more common. A variety of polymers may be used in the process; low and high-density polyethylene and polypropylene are the most popular.

Furthermore, the BFS process flow is normally impacted by only two raw materials—product and polymer—that are each processed inline, thereby making the process amenable to large uninterrupted batch sizes, some in excess of 500,000 or 1,000,000 units, and fill durations of up to 120 hours.

In a typical operation, to form the container, thermoplastic is continuously extruded in a tubular shape. When the tube reaches the proper length, the mould closes and the parison is cut. The bottom of the parison is pinched, closed and the top is held in place with a set of holding jaws. The mould is then transferred to a position under the filling station. To fill the container, the nozzle assembly lowers into the parison until the nozzles form a seal with the neck of the mould. Container formation is completed by applying vacuum on the mould side of the container and by blowing sterile filtered air into the interior of the container. The fill system delivers a precise dosage of product into the container. The nozzles retract into their original position. Lastly, to seal the container, following completion of the filling process, the top of the container remains semi-molten. Separate seal moulds close to form the top and hermetically seal the container. The moulds open and the container is then conveyed out of the machine. The whole of the above process is operated in pharmaceutical aseptic processing conditions.

BFS machines are commercially available from a number of suppliers, including Weiler Engineering, Inc (US) and rommelag USA Inc (US).

The invention is now illustrated in the following non-limiting examples.

### EXAMPLES

### Example 1

A bulk nebulizer formulation was prepared having the following composition:

| Component | Amount/Concentration | |
|---|---|---|
| Levalbuterol | 0.625 | mg/ml |
| Ipratropium | 0.25 | mg/ml |
| NaCl solution (for injection) | 9 | mg/ml |

| | |
|---|---|
| HCl | To adjust pH to 4.0 |

The formulation was loaded into 2 ml (extractable volume), twist-top plastic ampoules.

### Example 2

A bulk nebulizer formulation was prepared having the following composition:

| Component | Amount/Concentration |
|---|---|
| Levalbuterol | 0.83 mg/ml |
| Ipratropium | 0.33 mg/ml |
| NaCl solution (for injection) | 9 mg/ml |
| HCl | To adjust pH to 4.0 |

The formulation was loaded into 1.5 ml (extractable volume), twist-top plastic ampoules.

The invention hence provides nebulizer formulations and uses thereof.

1. Use of a formulation comprising levalbuterol and ipratropium in a pharmaceutically acceptable carrier in the manufacture of a medicament for the treatment of COPD wherein the medicament is suitable for administration via a nebuliser ampoule containing not more that 2.2 ml of said formulation.

2. The use of claim 1, wherein the ampoule contains not less than 0.5 ml of said formulation.

3. The use of claim 1, wherein the ampoule contains about 1.0 to 2 ml of said formulation.

4. The use of claim 1, wherein the ampoule contains about 1.5 ml to 2 ml of said formulation.

5. Use of a formulation comprising levalbuterol and ipratropium in a pharmaceutically acceptable carrier in the manufacture of a medicament for the treatment of COPD or asthma.

6. The use of claim 5, wherein the patient has asthma.

7. The use of claim 5, wherein the patient has COPD.

8. The use of claim 5, wherein the formulation contains about 1.25 milligrams of levalbuterol.

9. The use of claim 5, wherein the formulation contains about 0.5 milligrams of ipratropium.

10. A filled ampoule containing a formulation comprising levalbuterol and ipratropium, and a pharmaceutically acceptable carrier.

11. The filled ampoule of claim 10, containing 2.2 ml or less of said formulation.

12. The filled ampoule of claim 10, containing 2.0 ml or less of said formulation.

13. The filled ampoule of claim 10, containing about 1.0 to 2 ml of said formulation.

14. Use of a formulation comprising levalbuterol and an anticholinergic agent in a pharmaceutically acceptable carrier in the manufacture of a medicament for increasing patient compliance in use of a nebuliser formulation wherein the medicament is suitable for administration via an ampoule containing not more than 2.2 ml and not less than 0.3 ml of said formulation.

15. The use of claim 14, wherein the ampoule contains not more than 2 ml and not less than 0.5 ml of said formulation.

16. The use of claim 14, wherein the formulation contains from 0.75 to 2.0 mg of levalbuterol.

17. The use of claim 14, wherein the formulation contains from 1.0 to 1.5 mg of levalbuterol.

18. The use of claim 14, wherein the formulation contains from 0.25 to 1.0 mg of ipratropium.

19. The use of claim 14, wherein the formulation contains from 0.4 to 0.7 mg of ipratropium.

20. A kit comprising:
(1) a container, containing a single unit dose of 2.2 ml or less in volume of a therapeutically effective amount of levalbuterol and an anticholinergic agent, and a pharmaceutically acceptable carrier; and
(2) instructions on how the dose is to be used.

21. The kit of claim 20, wherein and the instructions explain that the single unit dose can be administered in less time than a previously known formulation.

22. The kit of claim 20, wherein the instructions explain that the patient should continue administering the dose until the complete dose has been administered.

23. The kit of claim 20, wherein the single unit dose is about 2.0 ml in volume.

24. The kit of claim 23, wherein the volume is about 1.5 ml.

25. The kit of claim 20, wherein the anticholinergic agent is selected from the group consisting of ipratropium, tiaproprium and cromolyn sodium.

26. The kit of claim 25, wherein the anticholinergic agent is ipratropium.

27. The kit of claim 20, comprising instructions to deliver the dose using a high efficiency nebuliser.

28. The kit of claim 27 wherein the nebuliser is a jet nebuliser.

29. The kit of claim 20, comprising at least 25 of said single unit doses.

30. The kit of claim 29, comprising at least 120 of said single unit doses.
